(19) United States
(12) Patent Application Publication
(10) Pub. No.: US 2024/0050621 A1
(43) Pub. Date: Feb. 15, 2024

(54) OCULAR SEALANTS AND METHODS OF USING THE SAME

(71) Applicants: Massachusetts Eye and Ear Infirmary, Boston, MA (US); The Regents of the University of California, Oakland, CA (US)

(72) Inventors: Reza Dana, Newton, MA (US); Nasim Annabi, Los Angeles, CA (US); Clotilde Jumelle, Somerville, MA (US); Ehsan Shirzaei Sani, Los Angeles, CA (US)

(21) Appl. No.: 17/766,445

(22) PCT Filed: Oct. 8, 2020
(86) PCT No.: PCT/US2020/054838

§ 371 Date: Apr. 4, 2022

Related U.S. Application Data
(60) Provisional application No. 62/912,617, filed on Oct. 8, 2019.

(51) Int. Cl. A61L 24/08; A61L 24/00; A61L 24/04

(52) U.S. Cl. CPC A61L 24/08; A61L 24/0031; A61L 24/043; A61L 2430/16

(57) ABSTRACT

The present disclosure presents sealant compositions which can be used in the treatment of an ocular surface injury and methods of treating an ocular surface injury in a subject in need thereof. The sealant compositions can include methacrylated hyaluronic acid (MeHA); GelMA; optionally, poly(ethylene glycol) diacrylate (PEGDA); and a visible light-activated photoinitiator. The methods include applying a sealant composition to an applicator; placing the applicator containing the sealant composition onto a surface of the eye of a subject, wherein the surface of the eye has or is suspected of having the ocular surface injury; and photo-crosslinking the sealant composition by exposing the applicator and the sealant composition to a visible light, for example, a visible light having a wavelength of about 400 nanometers (nm) to 800 nm.

### CLAIM OF PRIORITY

This application claims the benefit of U.S. Provisional Patent Application Ser. No. 62/912,617, filed on Oct. 8, 2019. The entire contents of the foregoing are hereby incorporated by reference.

### FEDERALLY SPONSORED RESEARCH

This invention was made with government support under grant number W81XWH-18-1-0654 awarded by the Department of Defense, grant number EB023052 awarded by the National Institutes of Health, and grant number HL140618 awarded by the National Institutes of Health. The government has certain rights in the invention.

### TECHNICAL FIELD

The present disclosure presents sealant compositions for use in the treatment of an ocular surface injury and methods of treating an ocular surface injury in a subject in need thereof with the sealant compositions. The sealant compositions can include methacrylated hyaluronic acid (MeHA); gelatin methacryloyl (GelMA); optionally, poly(ethylene glycol) diacrylate (PEGDA); and a visible light-activated photoinitiator. The methods include applying a sealant composition to an applicator; placing the applicator containing the sealant composition onto a surface of the eye of a subject, wherein the surface of the eye has or is suspected of having the ocular surface injury; and photo-crosslinking the sealant composition by exposing the applicator and the sealant composition to a visible light, e.g., a visible light having a wavelength of about 400 nanometers (nm) to 800 nm.

### BACKGROUND

With 1.5-2 million new cases annually worldwide, corneal injuries are common causes of corneal scarring and vision loss. In addition to injuries, infection and immune- and chemical-induced insults to the eye lead to millions of cases of corneal ulcers each year globally (e.g., best estimates are nearly 1 million clinic visits at a direct cost of $175 million per year in the US alone).

Surgery involving placement of sutures to close defects and tissue transplantation are common modalities considered standard of care for serious corneal defects and injuries. However, in cases of trauma, suturing nearly always induces irregular astigmatism. For complex injuries involving tissue loss and/or dense scar formation, transplantation is commonly employed. However, tissue grafting presents several disadvantages, such as the need for donor tissue (and associated high costs) and the ever-present risk of immune rejection. Moreover, both suturing and tissue transplantation are resource intensive and require advanced surgical skills. In cases where the cornea has thinned from inflammation, infection or injury, and where transplant surgery may not be required, surgical adhesives can be used, including cyanoacrylate glue (primarily used), fibrin glues, or polyethylene-glycol (PEG)-based sealants. However, none of these marketed adhesives are FDA-approved for: (i) filling corneal defects, or for (ii) sealing larger (e.g., greater than about 2 mm) corneal incisions or perforations.

### SUMMARY

Certain aspects of the present disclosure are directed to sealant compositions including methacrylated hyaluronic acid (MeHA); gelatin methacryloyl (GelMA); optionally, polyethylene glycol diacrylate (PEGDA); and a visible light-activated photoinitiator. In some embodiments, the sealant composition includes methacrylated hyaluronic acid (MeHA); GelMA; and a visible light-activated photoinitiator. In some embodiments, the sealant composition can be used for repair of an ocular surface injury or defect.

In some embodiments, the sealant composition does not comprise a hydrolyzing enzyme. In some embodiments, the sealant composition does not comprise a glycosidase hydrolyzing enzyme. In some embodiments, the visible light-activated photoinitiator comprises triethanolamine, N-vinylcaprolactam, riboflavin, 2-hydroxy-4′-(2-hydroxyethoxy)-2-methylpropiophenone, Eosin Y disodium salt, 4,6-trimethylbenzoylphosphinate, triethanol amine, dl-2,3-diketo-l,7,7-trimethylnorcamphane (CQ), 1-phenyl-1,2-propadione (PPD), 2,4,6-trimethylbenzoyl-diphenylphosphine oxide (TPO), bis(2,6-dichlorobenzoyl)-(4-propylphenyl)phosphine oxide, 4,4′-bis(dimethylamino)benzophenone, 4,4′-bis(diethylamino)benzophenone, 2-chlorothioxanthen-9-one, 4-(dimethylamino)benzophenone, phenanthrenequinone, ferrocene, diphenyl(2,4,6 trimethylbenzoyl)phosphine oxide/2-hydroxy-2-methylpropiophenone (50/50 blend), dibenzosuberenone, (benzene) tricarbonylchromium, resazurin, resorufin, benzoyltrimethylgermane, derivatives thereof, or any combination thereof. In some embodiments, the visible light-activated photoinitiator comprises a mixture of triethanolamine, N-vinylcaprolactam, riboflavin, 2-hydroxy-4′-(2-hydroxyethoxy)-2-methylpropiophenone, and Eosin Y disodium salt. In some embodiments, the visible light-activated photoinitiator is activated upon exposure of light having a wavelength between about 420 nanometers (nm) to 550 nm. In some embodiments, the MeHA comprises glycidyl methacrylate-hyaluronic acid. In some embodiments, the MeHA is present in the sealant composition at a concentration between about 1% and 3% weight per volume (w/v). In some embodiments, the GelMA comprises methacrylamide substitution and methacrylate substitution. In some embodiments, the ratio of methacrylamide substitution to methacrylate substitution is between about 80:20 and 99:1. In some embodiments, the GelMA is present in the sealant composition at a concentration between about 2% and 4% (w/v). In some embodiments, the GelMA is present in the sealant composition at a concentration between about 0.5% and 1% (w/v). In some embodiments, the GelMA has a degree of methacryloyl substitution between about 30% and 85%. In some embodiments, the sealant composition further comprises a therapeutic agent. In some embodiments, the therapeutic agent comprises an antibiotic, an anti-inflammatory drug, a growth factor, or any combination thereof. In some embodiments, the growth factor comprises epithelial growth factor, fibroblast growth factor, nerve growth factor, hepatocyte growth factor, or any combination thereof. In some embodiments, the sealant composition has a swelling ratio ranging from about 25% to about 35%. In some embodiments, the sealant composition has a viscosity ranging from about 0.5 Pascal-seconds (Pa·s) to about 200 Pa·s. In some embodiments, the sealant composition has a burst strength of about 100 millimeters of mercury (mmHg) to 150 mmHg. In some embodiments, the sealant composition has a degradation rate of about 30 days to 35 days. In some embodiments, the sealant composition is for use in a repair of an ocular surface injury. In some embodiments, PEGDA is present at a concentration between about 0.5% and 1% (w/v).

Certain aspects of the present disclosure are directed to methods of treating an ocular surface injury in an eye of a subject. The method can include applying any of the sealant compositions of the disclosure to an applicator; placing the applicator containing the sealant composition on a surface of the eye of the subject, wherein the surface has or is suspected of having the ocular surface injury; and photo-crosslinking the sealant composition by exposing the sealant composition to a visible light.

In some embodiments, the applicator is a contact lens. In some embodiments, the light has a wavelength of about 400 nanometers (nm) to 800 nm. In some embodiments, the ocular surface injury is a corneal or scleral injury. In some embodiments, the corneal injury is a corneal full-thickness laceration or a corneal full-thickness perforation. In some embodiments, the ocular surface injury has a depth that is greater than about 350 microns. In some embodiments, the ocular surface injury extends into a Descemet's membrane or a corneal endothelium. In some embodiments, the ocular surface injury is a full thickness laceration or a full thickness perforation.

The term “ocular surface injury,” as used herein, can include ulcers, lacerations, defects, perforations, or intentionally performed incisions (e.g., as is done in surgery) of the cornea or sclera.

The terms “subject” or “patient” as used herein refer to any mammal (e.g., a human or a veterinary subject, e.g., a dog, cat, horse, cow, goat, sheep, mouse, rat, or rabbit) to which a composition or method of the present disclosure may be administered, e.g., for experimental, diagnostic, prophylactic, and/or therapeutic purposes. The subject may seek or need treatment, require treatment, is receiving treatment, will receive treatment, or is under care by a trained professional for a particular disease or condition.

The term “sealant composition” as used herein can refer to a precursor sealant composition (e.g., a sealant composition before crosslinking polymerization) and/or a sealant gel composition (e.g., a sealant composition after crosslinking polymerization), as provided by the corresponding context of the disclosure.

Other definitions appear in context throughout this disclosure. Unless otherwise defined, all technical and scientific terms used herein have the same meaning as commonly understood by one of ordinary skill in the art to which the contents of the present disclosure belong. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety, insofar as the contents thereof do not conflict with the contents of the present disclosure. In case of conflict, the present specification, including definitions, will control.

Certain embodiments of the present disclosure include sealant compositions and methods of using sealant compositions for the treatment of ocular surface injuries or defects. There is currently a need for improved adhesives that can meet the necessary requirements to manage corneal injuries or defects, including reduced surgical costs, a reduction in postoperative visits, and a reduction in complications related to suboptimal compliance with expensive post-operative medications for patients and major expenses for payers. The sealant compositions and methods of using the sealant compositions of the present disclosure address the above-mentioned necessary requirements. In some embodiments, the methods of using the sealant compositions described herein can reduce and/or eliminate the costs of anesthesia, time, and/or personnel associated with the treatment of ocular injuries.

In some embodiments, the sealant compositions of the present disclosure are easy to apply with an easy-to-dispense system. For example, the sealant compositions of the disclosure can include light-activated adhesive biomaterials for managing ocular injuries without sutures that can be applied via a contact lens applicator to ensure thorough coverage. 95% of ocular drugs are topical and patient compliance is notoriously poor, affecting post-operative outcomes. In some embodiments, the sealant compositions enable sustained delivery of a therapeutic agent (e.g. anti-inflammatories, antibiotics, anti-glaucoma medications and pro-regenerative proteins). In some embodiments, the sealant compositions of the present disclosure permit the incorporation of drugs for better management of ocular injuries and defects. In some embodiments, thin applications of the sealant compositions (e.g., in a transparent formulation) can provide prolonged drug delivery even to non-injured eyes and can have ocular pharmacotherapy applications. In some embodiments, the methods and sealant compositions of the present disclosure can deliver a therapeutic agent locally to the ocular surface for one or more weeks without the need for a patient to manually apply the drug, which can prevent poor medication compliance and adverse effects caused by it.

In some embodiments, the sealant compositions have biocompatibility with ocular tissue. In some embodiments, the sealant compositions promote rapid sealing of ocular wounds. In some embodiments, the biomechanical properties of the sealant compositions are similar to the biomechanical properties of the native tissue (e.g., the cornea). In some embodiments, the sealant compositions have strong adhesion and high retention. Furthermore, in some embodiments the sealant compositions have a smooth surface once applied to a surface (e.g., an ocular surface). In some embodiments, the sealant compositions have the ability to permit controlled and sustained release of medications and/or therapeutics over a defined period of time.

Where values are described in the present disclosure in terms of ranges, endpoints are included. Furthermore, it should be understood that the description includes the disclosure of all possible sub-ranges within such ranges, as well as specific numerical values that fall within such ranges irrespective of whether a specific numerical value or specific sub-range is expressly stated.

Other features and advantages of the present disclosure will be apparent from the following detailed description and figures, and from the claims.

Various embodiments of the features of this disclosure are described herein. However, it should be understood that such embodiments are provided merely by way of example, and numerous variations, changes, and substitutions can occur according to those skilled in the art without departing from the scope of this disclosure. It should also be understood that various alternatives to the specific embodiments described herein are also within the scope of this disclosure.

### DESCRIPTION OF DRAWINGS

FIG. 1 is a schematic illustrating an example method of treating an ocular surface injury in an eye of a subject using an example sealant composition.

FIGS. 2A-2B are schematics illustrating the precursor gel preparation. FIG. 2A is a schematic illustrating an example methacrylation process of hyaluronic acid. FIG. 2B is a schematic illustrating an example photo-crosslinking process of a sealant composition.

FIGS. 3A-3D show physical characterization of an example sealant composition before and after photo-crosslinking. FIG. 3A shows images of example precursor sealant compositions prepared with different methacrylated hyaluronic acid (MeHA) concentrations. FIG. 3B is a graph showing the steady-shear viscosity of example precursor sealant compositions containing different MeHA/GelMA/PEGDA concentrations. FIG. 3C is a graph showing the shear stress of example precursor sealant compositions containing different MeHA/GelMA/PEGDA concentrations. FIG. 3D shows images of an example solid and transparent MeHA sealant after photo-crosslinking.

FIGS. 4A-4B show assessment of the in vitro biocompatibility of an example sealant composition after photo-crosslinking. FIG. 4A shows microscopic images of human corneal epithelial cells (HCECs) incubated for 24 h with culture medium or fluid extracts of photo-crosslinked hydrogels prepared with different concentrations of HAGM, GelMA and PEGDA and then stained with Hoechst 33342 (blue—Nuclei dye), calcein-AM (green—viable dye) and propidium iodide (red—dead dye) (n=3 per group). FIG. 4B is a graph showing the optical density (OD) measured at 570 nm (690 nm background absorbance subtracted). Data is represented as mean±standard deviation (SD) (n=6 per group).

FIGS. 5A, 5B, and 5C show the mechanical characterization of example sealant compositions formed at different ratios of hyaluronic acid glycidyl-methacrylate (MeHA), gelatin methacryloyl (GelMA), and (polyethylene glycol) diacrylate (PEGDA). FIG. 5A is a graph showing the elastic modulus, measured in kilopascals (kPa), of the example sealant compositions. FIG. 5B is a graph showing the ultimate tensile strength (i.e., ultimate stress) measured in kPa of the example sealant compositions. FIG. 5C is a graph showing the percent extensibility of the example sealant compositions. The example sealant compositions were formed after 4 minutes of visible light exposure time. Data is represented as mean±SD (*p<0.05, **p<0.01, ***p<0.001, ****p<0.0001 and n≥3).

FIGS. 6A and 6B show the hydrogel properties of example sealant compositions. Swelling ratios and water content of the example sealant compositions are shown in FIGS. 6A and 6B, respectively. **p<0.005 compared with H3, ##p<0.005 compared with H3G2.

FIG. 7 shows the degradation assessment of example sealant compositions. Degradation rate (n=6) of formulation composed of MeHA sealant only or in combination with GelMA and PEGDA at different concentrations. Graph is presented as mean±SD. **p<0.005 compared with H3, ##p<0.005 compared with H3P1.

FIGS. 8A-8C show adhesion assessment of example sealant compositions on an ex vivo pig corneal injury. FIG. 8A shows representative images for sealant composition application. FIG. 8B shows optical coherent tomography (OCT) images of an example sealant composition after photo-crosslinking before and after applicator removal. FIG. 8C is a graph showing the average burst pressure of an example sealant composition (n=10) compared with a commercially available optical sealant, ReSure® (Control, n=5). Graph is presented as mean±standard error of the mean (SEM). (**p<0.005 compared with H3.)

FIGS. 9A and 9B show in vitro kinetics release of HGF from different sealants. FIG. 9A is a graph showing HGF concentrations in picograms/milliliter (pg/ml) released by different formulations at different time points (n=6 per group). Graph is presented as mean±standard error of the mean (SEM). FIG. 9B shows Area Under the Curve (AUC), Tₘₐₓ, and Cₘₐₓ of HGF released according to the formulation.

### DETAILED DESCRIPTION

There is an unmet need for better management of corneal injuries, corneal ulcers from severe infections, scleral injuries, corneal perforations, or other corneal defects. The use of cyanoacrylate and fibrin glues, used off-label as described herein, is largely ineffective in closing defects and complex lacerations. Fibrin glues are ineffective for “wet” leaking and complex wounds. Cyanoacrylate glues have many limitations including toxicity, opaqueness, difficulty in application, roughness, and lack of biocompatibility.

The sealant compositions of the present disclosure meet one or more of the following requirements, which are not met by sealant compositions currently used in the art: 1) easy application with an easy-to-dispense system; 2) biocompatibility; 3) rapid sealing of wounds; 4) biomechanical properties similar to the native-tissue cornea; 5) strong adhesion and high retention; and 6) a smooth surface once applied. In addition, the sealant compositions of the present disclosure permit 7) controlled and sustained release of medications/therapeutics over a defined period of time. As such, the sealant compositions of the present disclosure present clear and unexpected improvements in the management of ocular injuries or defects.

### Sealant Compositions

The present disclosure presents sealant compositions which include adhesive biomaterials (e.g., hydrogels) that can include one or more of a chemically modified hyaluronic acid (HA), a chemically modified gelatin, and a chemically modified polyethylene glycol (PEG). In some embodiments, the sealant composition includes MeHA, GelMA, PEGDA, and a visible light-activated photoinitiator. In some embodiments, the sealant composition includes MeHA, GelMA, and a visible light-activated photoinitiator. In some embodiments, the sealant compositions can include MeHA, GelMA, or a combination of both to meet specific medical applications (e.g., closure of complex full-thickness lacerations). In some embodiments, the sealant composition does not include a hydrolyzing enzyme (e.g., glycosidase). In some embodiments, the sealant composition is a hydrogel. In some embodiments, the sealant composition is biocompatible. In some embodiments, the sealant composition is non-toxic to an ocular environment. In some embodiments, the sealant composition is a photocrosslinkable, viscoelastic, composite hydrogel.

Hyaluronic acid (HA) is a viscoelastic and highly biocompatible glycosaminoglycan, that is naturally present in the cornea. HA is known to play a role in the regeneration and reconstruction of soft tissues. In some embodiments, a chemically modified HA can be included in the sealant compositions of the present disclosure. In some embodiments, the chemically modified HA can be methacrylated hyaluronic acid or a photocrosslinkable derivative of HA. In some embodiments, methacrylation of HA can be performed by ring opening of the HA backbone reaction and a reversible transesterification reaction. In some embodiments, the methacrylated hyaluronic acid included in the sealant composition is glycidyl methacrylate-hyaluronic acid (MeHA).

In some embodiments, the MeHA is present at a concentration between about 1% and about 3% weight per volume (w/v) in the sealant composition. In some embodiments, the sealant composition includes MeHA at a concentration of about 0.5% w/v. In some embodiments, the sealant composition includes MeHA at a concentration of about 1% w/v. In some embodiments, the sealant composition includes MeHA at a concentration of about 2% w/v. In some embodiments, the sealant composition includes MeHA at a concentration of about 3% w/v. In some embodiments, the sealant composition includes MeHA at a concentration of about 4% w/v. In some embodiments, the sealant composition includes MeHA at a concentration of about 5% w/v. In some embodiments, the sealant composition includes methacrylated hyaluronic acid at a concentration ranging from about 0.1% to about 20% w/v, about 1% to about 15% w/v, about 2% to about 10% w/v, or about 3% to about 5% w/v. In some embodiments, the concentration of PEGDA is less than about 10% w/v, about 5% w/v, about 3% w/v, about 2% w/v, or about 1% w/v. In some embodiments, the concentration of PEGDA is greater than 0.5% w/v, about 1% w/v, about 2% w/v, about 3% w/v, about 4% w/v, about 5% w/v, about 6% w/v, or about 7% w/v. In some embodiments, the sealant composition includes PEGDA at a concentration of about 0.5% w/v or about 1% w/v.

Gelatin is a derivative from collagen which is the main structural component of the cornea. Gelatin has strong adhesive properties to cells and tissue due to the presence of RGD motifs in gelatin, a denatured form of collagen that is chemically modified to form a light-activated precursor. In some embodiments, a chemically modified gelatin can be included in the sealant compositions of the present disclosure. In some embodiments, the chemically modified gelatin can be modified with methacryloyl anhydride (MA) to form GelMA, a photocrosslinkable derivative of gelatin. In some embodiments, chemical modification of gelatin can be performed by a synthesis reaction of gelatin with methacrylic anhydride (MAA).

In some embodiments, the sealant composition includes GelMA with a degree of methacryloyl substitution (i.e., methacryloyl functionalization) ranging from at least about 30% to about 85%. In some embodiments, the sealant composition includes GelMA with a degree of substitution that can range from 30%-40%, 40%-50%, 50%-60%, 60%-70%, 70%-80%, or 80%-85%. In some embodiments, the degree of substitution of GelMA is greater than 10%, 20%, 30%, 40%, or 50%. In some embodiments, the degree of substitution of GelMA is less than 100%, 90%, 80%, 70%, or 60%. In some embodiments, the sealant composition includes GelMA with a degree of substitution of about 70%. In some embodiments, the GelMA includes methacrylamide substitution and methacrylate substitution. In some embodiments, the ratio of methacrylamide substitution to methacrylate substitution is between about 80:20 and 99:1. In some embodiments, the ratio of methacrylamide substitution to methacrylate substitution can range from 80:20 to 85:15, 85:25 to 90:10, 90:10 to 95:5, or 95:5 to 99:1.

In some embodiments, the concentration of GelMA in the sealant composition can range from about 2% to about 4% w/v. In some embodiments, the sealant composition includes GelMA at a concentration of about 0.5% w/v. In some embodiments, the sealant composition includes GelMA at a concentration of about 1% w/v. In some embodiments, the sealant composition includes GelMA at a concentration of about 2% w/v. In some embodiments, the sealant composition includes GelMA at a concentration of about 3% w/v. In some embodiments, the sealant composition includes GelMA at a concentration of about 4% w/v. In some embodiments, the sealant composition includes GelMA at a concentration ranging from about 0.1% to about 20% w/v, about 1% to about 15% w/v, about 2% to about 10% w/v, or about 3% to about 5% w/v. In some embodiments, the concentration of PEGDA is less than about 10% w/v, about 5% w/v, about 3% w/v, about 2% w/v, or about 1% w/v. In some embodiments, the concentration of PEGDA is greater than about 0.5% w/v, about 1% w/v, about 2% w/v, about 3% w/v, about 4% w/v, about 5% w/v, about 6% w/v, or about 7% w/v. In some embodiments, the sealant composition includes PEGDA at a concentration of about 0.5% w/v or about 1% w/v.

PEG is a synthetic polymer that is well-tolerated in the human body. Contrary to naturally derived polymers, PEG is not degradable and can allow a longer retention of the sealant composition in the body. In some embodiments, a chemically modified PEG can be included in the sealant compositions of the present disclosure. In some embodiments, the chemically modified PEG can include PEGDA, a photocrosslinkable derivative of PEG. In some embodiments, PEGDA can be synthesized by chemically reacting PEG with acryloyl chloride.

In some embodiments, the concentration of PEGDA in the sealant composition can range from about 0.5% to about 1% w/v. In some embodiments, the sealant composition includes PEGDA at a concentration of about 0.5% w/v. In some embodiments, the sealant composition includes PEGDA at a concentration of about 1% w/v. In some embodiments, the sealant composition includes PEGDA at a concentration ranging from about 0.01% to about 0.5% w/v, about 0.5% to about 1% w/v, about 1% to about 1.5% w/v, or about 1.5% to about 3% w/v. In some embodiments, the concentration of PEGDA is less than about 10% w/v, about 5% w/v, about 3% w/v, or about 1% w/v. In some embodiments, the concentration of PEGDA is greater than about 0.5% w/v, about 1% w/v, about 2% w/v, about 3% w/v, about 4% w/v, about 5% w/v, about 6% w/v, or about 7% w/v.

In some embodiments, the sealant composition can include MeHA and PEGDA. In some embodiments, the sealant composition includes about 3% MeHA and about 0.5% PEGDA. In some embodiments, the sealant composition includes about 3% MeHA and about 1% PEGDA. In some embodiments, the sealant composition includes about 3% MeHA and about 2% PEGDA.

In some embodiments, the sealant composition can include MeHA and GelMA. In some embodiments, the sealant composition includes about 3% MeHA and about 1% GelMA. In some embodiments, the sealant composition includes about 3% MeHA and about 2% GelMA. In some embodiments, the sealant composition includes about 3% MeHA and about 3% GelMA. In some embodiments, the sealant composition includes about 3% MeHA and about 4% GelMA.

In some embodiments, the sealant composition can include MeHA, PEGDA, and GelMA. In some embodiments, the sealant composition includes about 3% MeHA, about 0.5% PEGDA, and about 4% GelMA. In some embodiments, the sealant composition includes about 3% MeHA, about 1% PEGDA, and about 4% GelMA. In some embodiments, the sealant composition includes about 3% MeHA, about 0.5% PEGDA, and about 2% GelMA. In some embodiments, the sealant composition includes about 3% MeHA, about 1% PEGDA, and about 2% GelMA. In some embodiments, the sealant composition includes about 3% MeHA, about 0.5% PEGDA, and about 4% GelMA. In some embodiments, the sealant composition includes about 3% MeHA, about 1% PEGDA, and about 4% GelMA. In some embodiments, the sealant composition includes about 3% MeHA, about 0.5% PEGDA, and between about 2% to about 4% GelMA. In some embodiments, the sealant composition includes about 3% MeHA, about 1% PEGDA, and between about 2% to about 4% GelMA. In some embodiments, the sealant composition includes about 3% MeHA, between about 0.5 to about 1% PEGDA, and between about 2% to about 4% GelMA.

In some embodiments, the sealant compositions can include a photoinitiator that can be used to activate polymerization and solidification of the sealant composition when it is in a non-solid (e.g., viscous liquid, gel, or liquid) state. In some embodiments, the light exposure activates the photoinitiator, forming free-radicals, resulting in vinyl-bond crosslinking between methacrylate groups, and thus solidification of the sealant composition.

Different types of light source can be used to photo-crosslink the precursor gel sealant. Non-limiting examples of light sources that can be used to polymerize the sealant composition include visible light sources (e.g., white or blue light), ultraviolet light sources, near-infrared light sources, and fluorescent light sources. In some embodiments, the visible light-activated photoinitiator can be activated upon exposure of light having a wavelength between about 420 nanometers (nm) to 550 nm. In some embodiments, the visible light-activated photoinitiator can be activated upon exposure of light having a wavelength of about 460 nm. In some embodiments, the visible light-activated photoinitiator can be activated upon exposure of light having a wavelength ranging from about 400 nm to 800 nm. In some embodiments, the visible light-activated photoinitiator can be activated upon exposure of light having a wavelength less than 800, 750, 700, 650, 600, 550, 500, 450, or 400 nm. In some embodiments, the visible light-activated photoinitiator can be activated upon exposure of light having a wavelength greater than 400, 450, 500, 550, or 600 nm.

In some embodiments, the photoinitiator includes a light-activated photoinitiator. In some embodiment, the light-activated photoinitiator includes an ultraviolet light-activated photoinitiator. In some embodiment, the light-activated photoinitiator includes a near-infrared (NIR) light-activated photoinitiator. In some embodiment, the light-activated photoinitiator includes a visible light-activated photoinitiator. For example, the light-activated photoinitiator can includes a blue light-activated photoinitiator. In some embodiments, the visible light-activated photoinitiator includes triethanolamine, N-vinylcaprolactam, riboflavin, 2-hydroxy-4′-(2-hydroxyethoxy)-2-methylpropiophenone, Eosin Y disodium salt, 4,6-trimethylbenzoylphosphinate, triethanol amine, dl-2,3-diketo-l,7,7-trimethylnorcamphane (CQ), 1-phenyl-1,2-propadione (PPD), 2,4,6-trimethylbenzoyl-diphenylphosphine oxide (TPO), bis(2,6-dichlorobenzoyl)-(4-propylphenyl)phosphine oxide, 4,4′-bis(dimethylamino)benzophenone, 4,4′-bis(diethylamino)benzophenone, 2-chlorothioxanthen-9-one, 4-(dimethylamino)benzophenone, phenanthrenequinone, ferrocene, diphenyl(2,4,6 trimethylbenzoyl)phosphine oxide/2-hydroxy-2-methylpropiophenone (50/50 blend), dibenzosuberenone, (benzene) tricarbonylchromium, resazurin, resorufin, benzoyltrimethylgermane, derivatives thereof, or any combination thereof. In some embodiments, the visible light-activated photoinitiator includes a mixture of triethanolamine, N-vinylcaprolactam, riboflavin, 2-hydroxy-4′-(2-hydroxyethoxy)-2-methylpropiophenone, and Eosin Y disodium salt. In some embodiments, the visible light-activated photoinitiator comprises a mixture of two or more elements selected from triethanolamine, N-vinylcaprolactam, riboflavin, 2-hydroxy-4′-(2-hydroxyethoxy)-2-methylpropiophenone, and Eosin Y disodium salt.

In some embodiments, the sealant composition can include MeHA. In some embodiments, the sealant composition can include MeHA and a photoinitiator. In some embodiments, the sealant composition can include MeHA. In some embodiments, the sealant composition can include MeHA and a photoinitiator.

In some embodiments, the sealant composition can include MeHA and PEGDA. In some embodiments, the sealant composition can include MeHA, PEGDA, and a photoinitiator. In some embodiments, the sealant composition can include MeHA and PEGDA. In some embodiments, the sealant composition can include MeHA, PEGDA, and a photoinitiator.

In some embodiments, the sealant composition can include MeHA, PEGDA, and GelMA. In some embodiments, the sealant composition can include MeHA, PEGDA, GelMA, and a photoinitiator. In some embodiments, the sealant composition can include MeHA, PEGDA, and GelMA. In some embodiments, the sealant composition can include MeHA, PEGDA, GelMA, and a photoinitiator.

In some embodiments, the sealant composition can include MeHA and GelMA. In some embodiments, the sealant composition can include MeHA GelMA, and a photoinitiator. In some embodiments, the sealant composition can include MeHA and GelMA. In some embodiments, the sealant composition can include MeHA, GelMA, and a photoinitiator.

### Physical Properties of Sealant Composition

The physical properties of the sealant compositions of the disclosure, including but not limited to stiffness, elasticity, degradation rate, adhesion, and swelling, can be finely tuned by modulating the concentration of one or more of the polymers (e.g., MeHA, GelMA, PEGDA, and/or photoinitiator). Alternatively, or in combination to the polymer concentration modulation, the physical properties of the sealant can also be finely tuned by controlling the light exposure time (i.e., the polymerization time). In some embodiments, the sealant composition is exposed to a light source for about 4 minutes. In some embodiments, the sealant composition is exposed to a light source for about a period ranging from about 15 seconds to 15 minutes. In some embodiments, the sealant composition is exposed to a light source for about a period ranging from about 1 to 10 minutes. In some embodiments, the sealant composition is exposed to a light source for about 30 seconds to 1 minute, 1 to 2 minutes, 2 to 3 minutes, 3 to 4 minutes, 4 to 5 minutes, 5 to 6 minutes, 6 to 7 minutes, 7 to 8 minutes, 8 to 9 minutes, or 9 to 10 minutes. In some embodiments, the sealant composition is exposed to a light source for less than about 20, 15, 10, or 7 minutes. In some embodiments, the sealant composition is exposed to a light source for more than about 10 seconds, 30 seconds, 1, 3, or 5 minutes.

Treatment of ocular penetrating injuries (e.g., lacerations) is very challenging due to leakage of intraocular fluid from the injury site. In some embodiments, the sealant composition is a viscous gel that can retain its shape and/or consistency on an ocular injury site without running-off and is able to stop intraocular fluid leaking through the injury site. The viscosity of the sealant is an important property that allows the sealant to have a good retention (i.e., no run-off) on the surface of a cornea (e.g., when treating a corneal injury). In some embodiments, once polymerized and solidified, the sealant composition has a viscosity that is greater than the viscosity of the precursor sealant composition prior to photo-crosslinking and solidification. In some embodiments, the precursor sealant composition (e.g., the sealant prior to photo-crosslinking and solidification) has a viscosity that is greater than the viscosity of water. In some embodiments, the precursor sealant composition has a viscosity that is similar to the viscosity of toothpaste. In some embodiments, the sealant composition has a viscosity ranging from about 0.5 Pascal-seconds (Pa·s) to about 300 Pa·s. In some embodiments, the sealant composition has a viscosity of about 100 Pa·s at a low shear rate (e.g., at a shear rate of about 0.001 inverse seconds (s⁻¹) to 1 s⁻¹. In some embodiments, the sealant composition has a shear stress ranging from about 1 to 10 Pa at a low shear rate (e.g., at a shear rate of about 0.001 to 0.1 s⁻¹. In some embodiments, the sealant composition includes about 3% MeHa, and the precursor sealant composition has a viscosity of between about 30 Pa·s to about 300 Pa·s at a low shear rate (e.g., at a shear rate of about 0.001 inverse seconds (s⁻¹) to 1 s⁻¹. In some embodiments, the viscosity is about 100 Pa·s. In some embodiments, the sealant composition includes about 3% MeHa and about 4% GelMA, and the precursor sealant composition has a viscosity of between about 30 Pa·s to about 300 Pa·s at a low shear rate (e.g., at a shear rate of about 0.001 inverse seconds (s⁻¹) to 1 s⁻¹. In some embodiments, the viscosity is about 100 Pa·s. In some embodiments, the sealant composition includes about 3% MeHa, and the precursor sealant composition has a shear stress ranging from about 0.1 to 10 Pa at a low shear rate (e.g., at a shear rate of about 0.001 to 0.1 s⁻¹). In some embodiments, the sealant composition includes about 3% MeHa and about 4% GelMA, and the precursor sealant composition has a shear stress ranging from about 0.1 to 10 Pa at a low shear rate (e.g., at a shear rate of about 0.001 to 0.1 s⁻¹).

Important physical properties of the sealant composition are elastic modulus, ultimate stress (or ultimate tensile strength), and extensibility. In some embodiments, the sealant composition has an elastic modulus of about 25 kPa. In some embodiments, the sealant composition has an elastic modulus ranging from about 10 kPa to about 30 kPa. In some embodiments, the sealant composition has an elastic modulus ranging from about 20 kPa to about 30 kPa. In some embodiments, the sealant composition has an elastic modulus ranging from about 10 kPa to about 25 kPa. In some embodiments, the sealant composition has an elastic modulus ranging from about 15 kPa to about 25 kPa. In some embodiments, the sealant composition has an elastic modulus ranging from about 20 kPa to about 25 kPa. In some embodiments, the sealant composition includes about 4% GelMA, and the sealant gel composition has an elastic modulus ranging from between about 10 kPa to about 30 kPa, about 15 kPa to about 30 kPa, about 20 kPa to about 30 kPa, about 10 kPa to about 25 kPa, about 15 kPa to about 25 kPa, or about 20 kPa to about 25 kPa. In some embodiments, the sealant composition has an elastic modulus ranging from about 20 kPa to about 25 kPa. In some embodiments, the sealant composition includes about 3% MeHa and about 4% GelMA, and the sealant gel composition has an elastic modulus ranging from between about 10 kPa to about 30 kPa, about 15 kPa to about 30 kPa, about 20 kPa to about 30 kPa, about 10 kPa to about 25 kPa, about 15 kPa to about 25 kPa, or about 20 kPa to about 25 kPa. In some embodiments, the sealant composition includes about 3% MeHa, about 4% GelMA, and between about 0.5% to about 1% PEGDA, and the sealant gel composition has an elastic modulus ranging from between about 10 kPa to about 30 kPa, about 15 kPa to about 30 kPa, about 20 kPa to about 30 kPa, about 10 kPa to about 25 kPa, about 15 kPa to about 25 kPa, or about 20 kPa to about 25 kPa.

In some embodiments, the sealant composition has an ultimate stress of about 18 kPa. In some embodiments, the sealant composition has an ultimate stress ranging from about 5 kPa to about 20 kPa. In some embodiments, the sealant composition has an ultimate stress ranging from between about 10 kPa to about 20 kPa. In some embodiments, the sealant composition has an ultimate stress ranging from about 15 kPa to about 20 kPa. In some embodiments, the sealant composition includes about 4% GelMA, and the sealant gel composition has an ultimate stress ranging from between about 5 kPa to about 20 kPa, about 10 kPa to about 20 kPa, or about 15 kPa to about 20 kPa. In some embodiments, the sealant composition includes about 3% MeHa, about 4% GelMA, and between about 0.5% to about 1% PEGDA, and the sealant gel composition has an ultimate stress ranging from between about 5 kPa to about 20 kPa, about 10 kPa to about 20 kPa, or about 15 kPa to about 20 kPa.

In some embodiments, the sealant composition has an extensibility of about 40%. In some embodiments, the sealant composition has an extensibility ranging from between about 30% to about 60%. In some embodiments, the sealant composition has an extensibility ranging from between about 40% to about 50%. In some embodiments, the sealant composition includes about 3% MeHa, and the sealant gel composition has an extensibility ranging from between about 30% to about 60%. In some embodiments, the sealant composition includes about 3% MeHa, about 4% GelMA, and about 1% PEGDA, and the sealant gel composition has an extensibility ranging from between about 40% to about 50%.

In some embodiments, the sealant composition is a hydrogel. A hydrogel includes a polymer network filled with an interstitial solvent (e.g., a fluid) which includes water. A hydrogel can change its volume by absorbing (e.g., when it swells) or expelling a solvent. The swelling ratio of a hydrogel is defined as the fractional increase in the weight of the hydrogel due to water absorption, as shown in Example 9. Typically, the swelling ratio depends on both the polymer/solvent and the elasticity of the polymer. If the polymer is too stiff or the affinity is too low, then the swelling is low or weak. In contrast, low elasticity and high affinity favor high swelling. In some embodiments, the sealant composition has a swelling ratio ranging from about 25% to about 35%. In some embodiments, the sealant composition has a swelling ratio from about 10% to about 15%, about 15% to about 20%, about 20% to about 25%, about 25% to about 30%, about 30% to about 35%, about 35% to about 40%, about 40% to about 45%, or about 45% to about 50%. In some embodiments, the sealant composition has a swelling ratio of less than about 50%, about 45%, about 40%, about 35%, or about 30%. In some embodiments, the sealant composition has a swelling ratio more than about 5%, about 10%, about 15%, about 20%, about 25%, about 30%, about 35% or about 40%. In some embodiments, the sealant composition has a short-term swelling ratio (i.e., a swelling ratio measured for a period of about 1 to 6 hours) of about 30%. In some embodiments, the sealant composition has a short-term swelling ratio (i.e., a swelling ratio measured for a period of about 1 to 6 hours) of between about 25% to about 40%. In some embodiments, the sealant composition has a mid-term swelling ratio (i.e., a swelling ratio measured for a period of about 1 to 3 days) of about 30%. In some embodiments, the sealant composition has a mid-term swelling ratio (i.e., a swelling ratio measured for a period of about 1 to 3 days) of between about 25% to about 40%. In some embodiments, the sealant composition has a long-term swelling ratio (i.e., a swelling ratio measured for a period of about 1 to 4 weeks) of about 35%. In some embodiments, the sealant composition has a long-term swelling ratio (i.e., a swelling ratio measured for a period of about 1 to 4 weeks) of between about 30% to about 40%.

In some embodiments, the sealant composition includes about 3% MeHa and between about 0.5% to about 1% PEGDA, and the sealant gel composition has a short-term swelling ratio of between about 25% to about 40%, a mid-term swelling ratio of between about 25% to about 40%, and/or a long-term swelling ratio of between about 25% to about 40%. In some embodiments, the sealant composition includes about 3% MeHa, about 4% GelMA, and between about 0.5% to about 1% PEGDA, and the sealant gel composition has a short-term swelling ratio of between about 25% to about 35%, a mid-term swelling ratio of between about 25% to about 35%, and/or a long-term swelling ratio of between about 25% to about 35%. In some embodiments, the sealant composition includes about 3% MeHa and between about 2% to about 4% GelMA, and the sealant gel composition has a short-term swelling ratio of between about 10% to about 20%, a mid-term swelling ratio of between about 10% to about 20%, and/or a long-term swelling ratio of between about 10% to about 20%.

In some embodiments, the sealant composition has a water content of about 94% or more. In some embodiments, the sealant composition has a water content ranging from about 94% to about 97%. In some embodiments, the sealant composition includes about 3% MeHa, and the sealant gel composition has a water content ranging from about 94% to about 97%. In some embodiments, the sealant composition includes about 3% MeHa, about 4% GelMA, and about 1% PEGDA, and the sealant gel composition has a water content ranging from about 94% to about 95%.

The degradation rate of the sealant composition can be controlled based on the concentration of one or more polymers added (e.g., MeHA, PEGDA, and/or GelMA). In some embodiments, the sealant composition has a degradation rate of about 35 days. In some embodiments, the sealant composition has a degradation rate ranging from about 1 day to about 40 days. In some embodiments, the sealant composition has a degradation rate ranging from about 1 to about 5 days, about 5 to about 10 days, about 10 to about 15 days, about 15 to about 20 days, about 20 to about 25 days, about 25 to about 30 days, about 30 to about 35 days, or about 35 to about 40 days. In some embodiments, the sealant composition has a degradation rate of less than about 80, about 60, about 55, about 50, about 45, about 40, about 35, or about 30 days. In some embodiments, the sealant composition has a degradation rate more than about 1, about 5, about 7, about 10, about 14, about 21, about 25, about 30, about 35, or about 40 days. In some embodiments, the sealant composition includes about 3% MeHa and between about 0.5% to about 1% PEGDA, and the sealant gel composition has a degradation rate ranging from about 10 to about 35 days, or about 15 to about 35 days. In some embodiments, the sealant composition includes about 3% MeHa, about 4% GelMA, and between about 0.5% to about 1% PEGDA, and the sealant gel composition has a degradation rate ranging from about 25 to about 35 days, or about 30 days or more.

In some embodiments, the sealant composition has high adhesive properties, especially in wet environments. Typically, to measure the adhesive strength of an optical sealant, an in vitro burst pressure test can be conducted in which a clinically representative incision is made in an ex vivo animal eye (e.g., a porcine eye). An infusion cannula can be placed inside the eye in order to reproduce the physiologic intraocular pressure. Once the incision is created, the optical sealant can be applied over the incision, and the intraocular pressure, required to rupture the sealant, can be measured. Such intraocular pressure can be defined as the “burst strength” or “burst pressure.” In some embodiments, the sealant composition has a burst strength of about 100 millimeters of mercury (mmHg) to about 150 mmHg. In some embodiments, the sealant composition has a burst strength of about 125 mmHg. In some embodiments, the sealant composition has a burst strength ranging from about 70 to about 80 mmHg, about 80 to about 90 mmHg, about 90 to about 100 mmHg, about 100 to about 110 mmHg, about 110 to about 120 mmHg, about 120 to about 130 mmHg, about 130 to about 140 mmHg, about 140 to about 150 mmHg, or about 150 to about 160 mmHg. In some embodiments, the sealant composition has a burst strength of less than about 200 mmHg, about 190 mmHg, about 180 mmHg, about 170 mmHg, about 160 mmHg, about 150 mmHg, about 140 mmHg, about 130 mmHg, or about 120 mmHg. In some embodiments, the sealant composition has a burst strength of more than about 70 mmHg, about 80 mmHg, about 90 mmHg, about 100 mmHg, about 110 mmHg, or about 120 mmHg. In some embodiments, the sealant composition includes about 3% MeHa, and the sealant gel composition has a burst strength of more than about 50 mmHg, or between about 50 mmHg to about 150 mmHg. In some embodiments, the sealant composition includes about 3% MeHa and about 4% GelMA, and the sealant gel composition has a burst strength of more than about 100 mmHg, or between about 100 mmHg to about 150 mmHg. In some embodiments, the sealant composition includes about 3% MeHa, about 4% GelMA, and between about 0.5% to about 1% PEGDA, and the sealant gel composition has a burst strength of more than about 140 mmHg, or between about 140 mmHg to about 150 mmHg.

In some embodiments, the sealant compositions of the present disclosure can include a therapeutic agent as a drug delivery payload. In some embodiments, the therapeutic agent can include an antibiotic or antibacterial agent, an anti-inflammatory agent, a growth factor, an anti-fungal, or any combination thereof. In some embodiments, the therapeutic agent is ciprofloxacin. As such, the sealant compositions described herein can be used to develop a range of “off the shelf” products for various indications. In some embodiments, a sealant composition, which includes a therapeutic agent has improved healing properties compared to a sealant composition without a therapeutic agent (e.g., it can reduce the time that it takes for an ocular injury to heal when treated with the sealant composition, as compared to treatment with other commercially available ocular sealants or to sealant compositions that do not include a therapeutic agent, for example). In some embodiments, drug released by the sealant composition can reduce the risk of inflammation and contamination following injury. In some embodiments, drug released by the sealant composition can promote wound healing.

In some embodiments, sealant compositions that are loaded with growth factors (e.g., recombinant growth factors) can improve wound healing. Non-limiting examples of growth factors include therapeutic agents or biologic agents, such as recombinant hepatocyte growth factor or recombinant nerve growth factor. Thus, in some embodiments, the sealant compositions can be pro-regenerative (not simply a sealant) technology. In some embodiments, the sealant compositions can be designed for sustained drug delivery in healthy eyes. For example, the sealant compositions can be an engineered biomaterial capable of being loaded with ciprofloxacin (i.e., an antibiotic) encapsulated in micelles. In some embodiments, the sealant composition has antimicrobial properties. In some embodiments, the sealant compositions can treat chronic conditions or elute drug(s) post-injury or in infected eyes to prevent and/or treat infections. In some embodiments, the sealant compositions are drug-loaded and can control inflammation, promote healing, or a combination thereof. In some embodiments, the sealant compositions contain recombinant growth factors and corticosteroid-loaded nanoparticles for promoting tissue regeneration, controlling inflammation, or a combination thereof.

Non-limiting examples of suitable antibiotics include gatifloxacin, daptomicin, tigecycline, telavancin, chloramphenicol, fusidic acid, bacitracin, rifampin, ethambutol, streptomycin, isoniazid, and all those comprised in the following antibacterial families: glicopeptides (including but not limited to teicoplanin, vancomycin, etc.), aminoglicosydes (including but not limited to, gentamycin, tobramycin, amikacin, netimicin, etc.), cephalosporins (including but not limited to cefazolin, cefoxitin, cefotaxime, cefuroxime, moxalactam, etc.), macrolids (including but not limited to erythromycin), oxazolidinones (including but not limited to linezolid), quinolones, polymixins, sulfonamides, tetracyclines and penicillins. Non-limiting examples of suitable anti-fungal agents include anti-fungal agents from the following groups: polyene antifungals, imidazole and triazole antifungals, allylamines, echinocandines, and griseofulvine. Non-limiting examples of suitable anti-inflammatory agents include a steroidal anti-inflammatory drug (e.g., prednisolone), a non-steroidal anti-inflammatory drug (e.g., bromfenac), an mTOR inhibitor, a calcineurin inhibitor, a synthetic or natural anti-inflammatory protein, antiproliferative drugs (e.g., dexamethasone, 5-fluorouracil, daunomycin, paclitaxel, curcumin, resveratrol, and mitomycin), methylprednisolone, prednisolone, hydrocortisone, fludrocortisone, prednisone, celecoxib, ketorolac, piroxicam, diclorofenac, ibuprofen, and ketoprofen, rapamycin, cyclosporin, and tacrolimus/FK-506. In some embodiments, the growth factor is epithelial growth factor, fibroblast growth factor, nerve growth factor, hepatocyte growth factor, or any combination thereof. Further non-limiting examples of suitable growth factors include transforming growth factors (TGFs) (e.g., beta transforming growth factors such as, TGF-β1, TGF-β2, TGF-β3), fibroblast growth factors (FGFs), platelet derived growth factors (PDGFs), epidermal growth factors (EGFs), connective tissue activated peptides (CTAPs), osteogenic factors, bone morphogenetic proteins (e.g., BMP-1, BMP-2, BMP-3, BMP-4, BMP-5, BMP-6, BMP-7, BMP-8, BMP-9); heparin-binding growth factors (e.g., fibroblast growth factor (FGF), epidermal growth factor (EGF), insulin-like growth factor (IGF)), Inhibins (e.g., Inhibin A, Inhibin B), growth differentiating factors (for example, GDF-1), and Activins (e.g.,, Activin A, Activin B, Activin AB), and biologically active analogs, fragments, and derivatives of such growth factors.

The release kinetics of a sealant composition loaded with a therapeutic agent can be controlled by adjusting the concentration of one or more of the polymers in the formulation (e.g., MeHA, PEGDA, and/or GelMA). In some embodiments, the sealant composition delivers a therapeutic agent at a maximum or peak concentration in about 1 day. In some embodiments, the sealant composition delivers a therapeutic agent at a maximum or peak concentration in about 2 days.

### Methods of Treatment

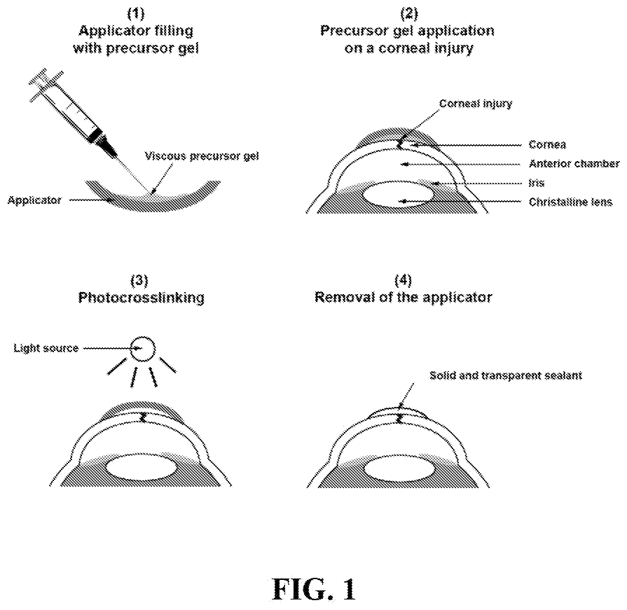

The present disclosure presents methods of treating an ocular surface injury (e.g., corneal or scleral injury) in an eye of a subject. In some embodiments, the present disclosure presents compositions for use in the treatment of an ocular surface injury (e.g., corneal or scleral injury) in an eye of a subject. As shown in FIG. 1, the methods can include the steps of applying the sealant to an applicator (e.g., a contact lens), contacting the applicator to the eye of the subject, and photo-crosslinking the sealant composition. The first step can include filling the applicator with the sealant composition. Once filled with the sealant composition, the applicator is directly applied on the corneal injury, e.g., by using forceps. This applicator can allow the operator to easily apply the precursor gel on ocular injuries with only forceps. The applicator containing the sealant composition can be inverted and placed on the surface of the eye of the subject having or suspected of having the ocular surface injury, without falling off or running off the surface of the applicator when inverted. Due to the high viscosity of the precursor sealant composition (e.g., a viscosity similar to the viscosity of toothpaste), leaking from the aqueous humor, for example, can be instantly halted when the applicator containing the sealant composition is placed on the ocular surface. The precursor sealant composition can be applied on any size or shape of ocular injuries to stop leaks from aqueous humor. In some embodiments, between about 20 and 200 microliters (μL ) of precursor sealant gel can be applied depending on the size and the shape of the ocular injury.

When the position of the applicator is satisfactory, the operator can initiate photo-crosslinking to solidify the sealant composition by using a visible light source. In some embodiments, the sealant composition can be photo-crosslinked by exposing the contact lens and the sealant composition to a visible light. In some embodiments, the visible light has a wavelength of about 400 nanometers (nm) to 800 nm. After photo-crosslinking, the sealant composition (e.g., an adhesive hydrogel) can become solid and transparent. Once the sealant composition is photo-crosslinked, the applicator can be removed from the ocular surface (e.g., by using forceps).

The applicator can be any suitable contact lens; for example, a hard contact lens, a soft contact lens, or a non-contact lens applicator that will permit controlled application of the sealant on the tissue. Non-limiting examples of contact lens types include rigid gas-permeable lenses and bandage lenses. The applicator can be a contact lens of different materials, diameters, base curve radiuses, power in diopters and central thickness. The applicator can also have a smooth and regular surface that comes in contact with the ocular surface thereby, limiting patient discomfort and vision loss.

In some embodiments, the sealant compositions can be applied as a drop (i.e., in a biomaterial precursor state) onto the eye without the need for an applicator. Exposure to visible light can permit crosslinking to provide an adhesive solid hydrogel with biomechanics analogous to the cornea. By adjusting the light exposure time, the polymerization of the adhesive compositions of the disclosure can be finely controlled, allowing for a precise application, as compared to commercially available ocular sealants.

In some embodiments, the sealant composition is used to prevent fluid leakage after cataract surgery. In some embodiments, the sealant composition remains localized over the incision, injury, and/or laceration to seal the wound and form a surface barrier. In some embodiments, upon photo-crosslinking, the precursor sealant composition becomes a solid and transparent hydrogel, forming a biocompatible and adhesive sealant on the ocular surface.

### Ocular Surface Injuries

The present disclosure presents methods and compositions for treating ocular surface injuries in an eye of a subject. Ocular surface injuries can include conjunctival laceration, corneal perforation, scleral perforation, incisions due to ocular surgery (e.g., cataract surgery) or any combination thereof. In some embodiments, the ocular surface injury is a corneal or scleral injury. Conjunctival laceration may occur following blunt or penetrating trauma. Conjunctival laceration is characterized with chemosis and subconjunctival hemorrhage. In such cases, it is important to rule out underlying scleral perforation. The fundus should be examined for any retinal tear or intraocular foreign body. An ultrasound may be done for the posterior segment evaluation.

Corneal lacerations and perforations represent approximately 1 in 10 of ocular traumatic injuries presenting in an emergency medical setting. Corneal lacerations and perforations can include partial thickness lacerations and full thickness lacerations. In addition, adnexal injuries, scleral perforation, or a combination thereof may be involved with corneal laceration and perforations. The standard of care for a corneal perforation include the removal of any contaminants in the wound area, repair of the tear, and maintenance of the watertight integrity of the ocular globe. Corneal perforation may also be associated with or caused by insertion of a foreign body. In some embodiments, the corneal injury is a corneal full-thickness laceration or a corneal full-thickness perforation. In some embodiments, the ocular surface injury is a full-thickness laceration or a full-thickness perforation. In some embodiments, the ocular surface injury is a full-thickness laceration or surgical incision or a full-thickness perforation. For example, the majority of ocular surgeries that require entry into the eye (e.g., cataract surgery) involve a full-thickness incision through the cornea or sclera. Current management protocols for full thickness lacerations including scleral wounds often require the use of sutures.

The sealant compositions of the disclosure can be used to treat ocular incisions or cuts or injuries having a length of less than about 1 mm to about 10 mm.

In some embodiments, the sealant compositions of the present disclosure can be used in the closure of full-thickness ocular defects and lacerations and in controlled and long-term drug elution. In some embodiments, indications can include post-operative applications of the biomaterial for drug elution in addition of closure of corneal ulcers, defects and perforations caused by a wide array of insults. The sealant compositions of the disclosure can be applied both under “normal” (e.g., in-the-office or operating room) settings, or under emergency “in-in-field” settings. Various providers, physicians, and, in select cases, physician assistants and paramedics (e.g., in the combat theater) can apply the sealant compositions described herein to seal the eye and elute drug(s) to heal defects. The sealant compositions described herein can circumvent many cases of transplants and patch grafts for corneal melts and defects.

### EXAMPLES

Certain embodiments of the present disclosure are further described in the following examples, which do not limit the scope of any embodiments described in the claims.

### Example 1—Synthesis of Poly(ethylene glycol) Diacrylates (PEGDA)

Poly(ethylene glycol) (PEG) was chemically modified with diacrylate to form PEGDA. To synthesize poly(ethylene glycol) diacrylates (PEGDA), poly(ethylene glycol) (PEG, Sigma Aldrich) was chemically reacted with acryloyl chloride (Sigma Aldrich). Accordingly, 10 grams of PEG was dissolved in 100 ml of dichloromethane (10% w/v) at 4° C. Next, triethylamine (Sigma Aldrich) was added to the PEG solution under N₂ environment. Acryloyl chloride (Sigma Aldrich) was then added to the solution and were dissolved in the PEG solution and stirred overnight under dry N₂ gas. The molar ratio of PEG, acryloyl chloride and triethylamine was 1:4:4. Finally, the insoluble salt (triethylamine-HCl) was filtered (using celite 545 powder and alumina column), and the product was precipitated by adding ice-cold ether. The crude product was filtered with 9 μm paper filter and dried in vacuum desiccator overnight to remove unreacted materials.

### Example 2 —Synthesis of Gelatin Methacryloyl (GelMA)

Gelatin was chemically modified with methacryloyl anhydride (MA) to form GelMA, a photocrosslinkable derivative of gelatin. Gelatin methacryloyl (GelMA) with 70% degree of substitution was synthesized. Briefly, 10% (w/v) gelatin from porcine skin (Sigma) solution in DPBS was reacted with 8 mL of methacrylic anhydride for 3 h. The solution was then dialyzed for 5 days to remove any unreacted methacrylic anhydride, and then placed in a −80° C. freezer for 24 h. The frozen polymer was then freeze-dried for 5 days.

### Example 3—Chemical Modification of Hyaluronic Acid (HA)

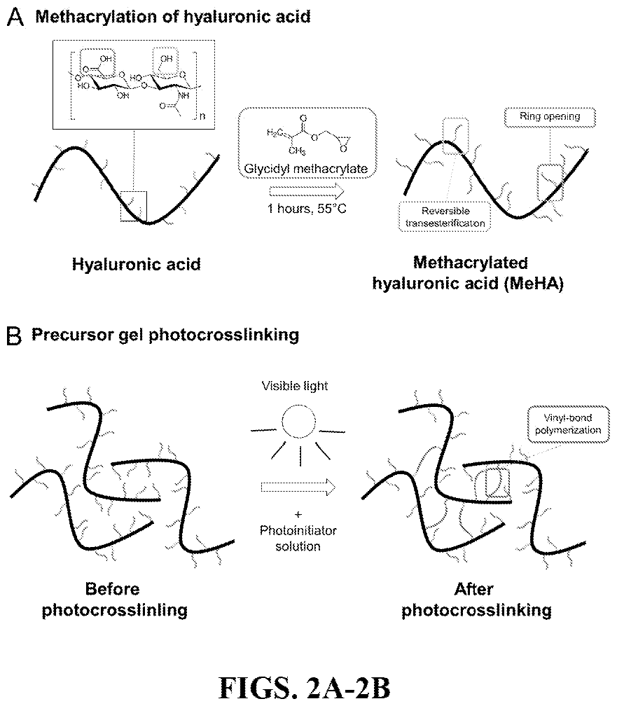

Hyaluronic acid (HA) was chemically modified with glycidyl methacrylate (GM) to form MeHA, a photocrosslinkable derivative of HA. The methacrylation of HA was performed by adding methacrylate groups to the HA backbone via ring opening and a reversible transesterification reaction (FIG. 2A). 2 g of hyaluronic acid sodium salt was dissolved in 200 ml deionized water overnight with continuous stirring. After it fully dissolved, 8.0 mL triethylamine, 8.0 mL glycidyl methacrylate, and 4.0 g of tetrabutyl ammonium bromide (TBAB) were added separately in the mentioned order, allowed to fully mix for 1 hour before the next addition. Following complete dissolution, the flask was then opened slightly and incubated at 55 ° C. for 1 hour. After cooling, the solution was then precipitated in 20 times excess acetone (4 L) as white solid fibers. The precipitate was then rinsed with fresh acetone, dissolved in 200 mL ultrapure water, and dialyzed for 2 days and lyophilized. The degree of methacrylation (DM) was calculated using proton nuclear magnetic resonance ('HNMR) analysis. First, HA and MeHA prepolymers were dissolved in Deuterium oxide (D₂O) (1% (w/v)). All spectra were run at room temperature by using a Bruker AV400 spectrometer (400 MHz). The DM is defined as the amount of methacrylate groups per one HA molecule repeat unit and was calculated from the ratio of the relative peak integrations of the methacrylate protons (peaks at ˜5.4, ˜5.7, and ˜1.8 ppm) and methyl protons (˜1.9 ppm) in HA molecule. According to this calculation method, a DM of 22.4±0.7% was found on the MeHA backbone. As shown in FIG. 2A, glycidyl-methacrylate hyaluronic acid (MeHA) was synthesized by exposure.

### Example 4—Preparation and Photo-Crosslinking of Composite Sealants

As shown in FIG. 2A, glycidyl-methacrylate hyaluronic acid (MeHA) was synthesized by reaction with glycidyl methacrylate for 1 hour at a temperature of about 55° C. MeHA, GelMA and/or PEGDA were dissolved in a photo-initiator (PI) solution that can crosslink polymers via visible light exposure (about 420-550 nm wavelength). The PI solution was prepared by dissolving 1.88% (w/v) triethanolamine (TEA) (Sigma), 1.25% (w/v) N-vinylcaprolactam (Sigma) and 0.5 mM Eosin Y disodium salt in phosphate-buffered saline (PBS). Different concentrations of MeHA, GelMA and/or PEGDA were then dissolved in the PI solution in order to study the effect of different concentrations/ratios for the repair of ocular injuries. Light exposure activated the PI, forming free-radicals, resulting in vinyl-bond crosslinking between methacrylate groups, and thus formation of a solid sealant (FIG. 2B). A light source emitting blue light (about 460 nm wavelength) was used for 4 min to crosslink the precursor gel.

### Example 5—Mechanical Characterization and Rheological Properties of the Adhesive Hydrogels

For tensile test, the biopolymers precursor solutions were prepared as described in Examples 1-4. Next, a 70 μL of each solution was placed into polydimethylsiloxane (PDMS) rectangular (14×5×1 mm) molds and photo-crosslinked via exposure to visible light (480-520 nm) for 240 seconds. After photo-crosslinking, the dimensions of the hydrogels were measured using digital calipers. The tensile tests were conducted using an Instron 5542 mechanical tester. Prior the test, the hydrogels were placed between two pieces of double-sided tape within the instrument tension grips and extended at a rate of 2 mm/min until failure. The slope of the stress-strain curves was obtained and reported as elastic modulus.

### Example 6—Viscous Properties of Sealant

Various formulations based on different concentrations and ratios of MeHA, GelMA and PEGDA were assessed (Table 1).

TABLE 1
Polymer concentrations and ratios
used for the different formulations

| Formulation | Polymer concentration (%) | | |
|---|---|---|---|
| name | MeHA (H) | PEGDA (P) | GelMA (G) |
| H1 | 1% | | |
| H2 | 2% | | |
| H3 | 3% | — | — |
| H3P0.5 | 3% | 0.5% | — |
| H3P1 | 3% | 1% | — |
| H3G2 | 3% | — | 2% |
| H3G4 | 3% | — | 4% |
| H3G4P0.5 | 3% | 0.5% | 4% |
| H3G4P1 | 3% | 1% | 4% |

The steady-shear viscosity for different concentrations of MeHA, GelMA, and/or PEGDA precursor gels was assessed.

For the rheological tests, different concentrations of bioadhesive precursors loaded between the parallel plates of an Anton-Paar 302 Rheometer. Steady shear viscosity assessment (frequency range: 0.01-1000 rad/s) were performed at a low strain of 1.0% for the solutions at 37° C. Steady shear rate sweeps were conducted by varying the shear rate from 0.01 to 1000 s⁻¹ to determine the viscosity of the prepolymer solutions.

At least 3 samples were tested for all experiments, and all data were expressed as mean±standard deviation (*p<0.05, **p<0.01, ***p<0.001 and ****p<0.0001). T-test, one-way, or two-way ANOVA followed by Tukey's test or Bonferroni test were performed where appropriate to measure statistical significance (GraphPad Prism 6.0, GraphPad Software).

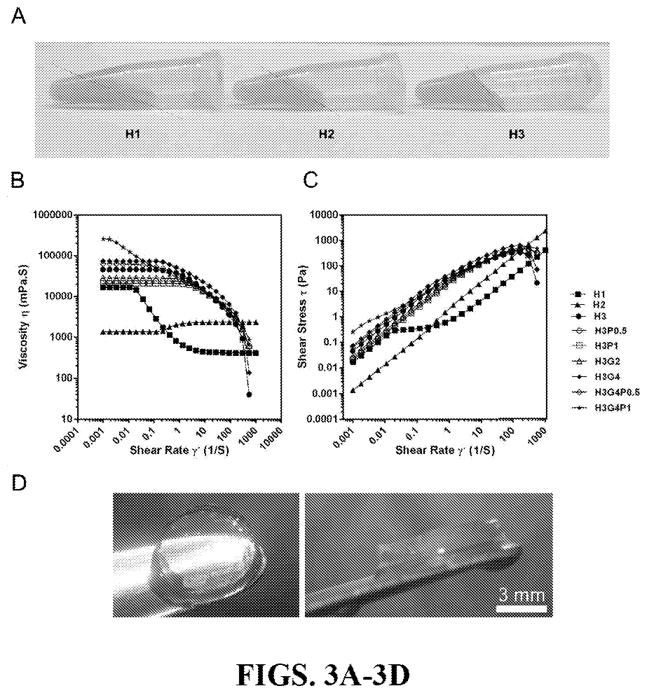

As expected, the results showed an increase of the viscosity of the prepolymer solutions, by increasing the total MeHA concentration (FIG. 3A-B). Generally, the precursor solutions containing 4% (w/v) GelMA showed a higher viscosity as compared to other formulations. Moreover, the 3% MeHA/1% PEGDA/4% GelMA precursor solution showed a significantly higher viscosity at low shear rates (<0.01 1/s) (FIG. 3B). Similar behavior was observed for shear stress values, where the shear stress of the prepolymer solutions increased significantly by increasing the total MeHA concentration, indicating prepolymer solutions with higher MeHA concentrations require higher injection force (FIG. 3C). There was no significant difference in the shear stress of the pre-polymer solutions containing 3% (w/v) MeHA. However, similar to the viscosity values, the 3% MeHA/1% PEGDA/4% GelMA precursor solution showed a higher shear stress at low shear rates (<0.01 1/s) (FIG. 3C). Based on the ex vivo preliminary experiments, formulations made with 3% MeHA were used in further studies based on the high viscosity limited the runoff, giving a good retention on the surface of the corneal injury.

### Example 7—In Vitro Toxicology of the MeHA Sealant

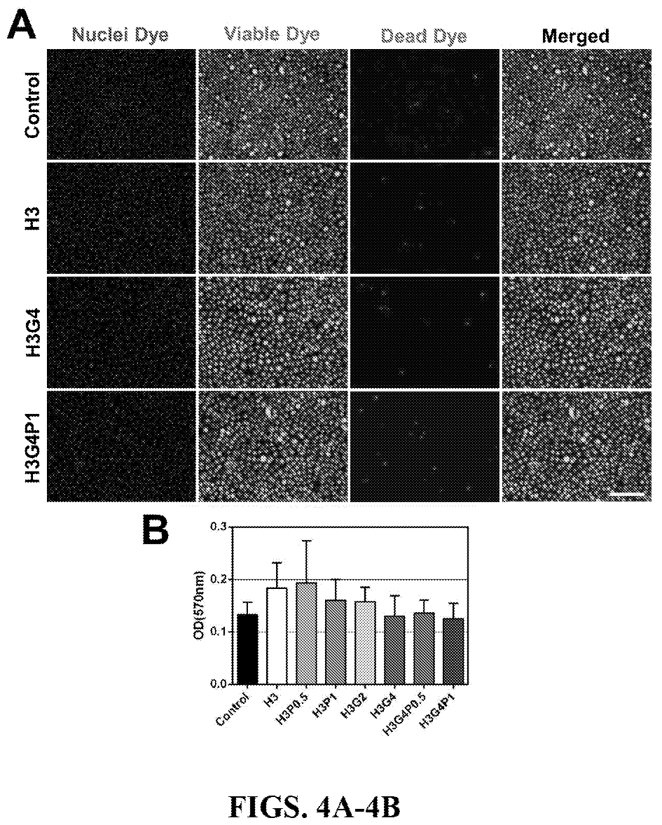

In vitro cytotoxicity tests were performed to assess cytocompatibility of the sealant using human corneal epithelial cells (HCECs). The elution test method was used according the ISO10993-1 Standard concerning the cytotoxicity of medical devices. Extracts were obtained by placing photo-crosslinked MeHA sealant samples in separate cell culture media at 37° C. for 24 hours (h). Fluid extracts were then applied to a confluent cell monolayer. Control groups were prepared similarly by incubating the cells with fresh medium. After 1 day of incubation at 37° C., cells were stained with calcein-AM and propidium iodide, which are markers of living and dead cells, respectively. Then, cells were observed using a bright field and fluorescence microscope. Results showed that no change was observed in the Viable/Dead staining and density between HCECs incubated with the culture medium alone or with the extracts of the photo-crosslinked hydrogels, regardless of the addition of GelMA or PEGDA (FIG. 4A). In vitro MTT-based toxicology assay (TOX-1, Sigma) was also performed on each group following manufacturer protocols (FIG. 4B). Results demonstrated no significant difference in optical density (OD) at 570 nm when measured between all groups tested. These results suggest that all photo-crosslinked hydrogels tested are not toxic for human cells and thus, could be well-tolerated on the human ocular surface.

### Example 8—Mechanical Properties of Several Sealant Formulations

### Sample Preparation

For each formulation shown in Table 1, 70 μL of precursor gel containing different concentrations of was pipetted into polydimethylsiloxane cylindrical molds having a diameter of about 6 mm and a height of about 2.5 mm. The precursor gels in the molds were then photo-crosslinked for about 4 min with a blue light source to form a solid sample, as shown in FIG. 3D.

### Tensile Tests

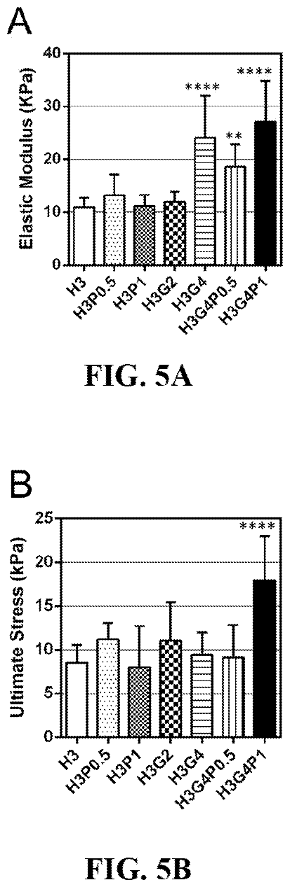

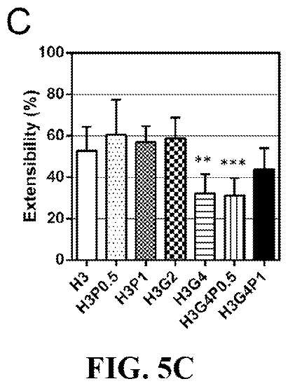

The tensile tests were conducted using a mechanical tensile tester (Instron® 5542). Prior to conducting the tensile tests, the samples (at least 3 per group) were placed between two pieces of double-sided tape within the instrument tension grips and extended at a rate of 2 mm/min until failure. The slope of the stress-strain curves was obtained and reported as elastic modulus. Tensile tests revealed that the elastic modulus of the adhesive hydrogels containing 4% (w/v) GelMA is significantly higher than those formed with lower GelMA content or without GelMA (FIG. 5A). The maximum ultimate stress (UTS) was observed for the 3% MeHA/1% PEGDA/4% GelMA hydrogel adhesives, which was significantly higher than the UTS for other gels. Moreover, there were no significant differences in the UTS values for all other compositions of the bioadhesive hydrogels (FIG. 5B). Furthermore, the extensibility of the composite adhesives was decreased significantly for the adhesive hydrogels with 4% (w/v) GelMA content, as compared to other compositions (FIG. 5C). This can be correlated to higher stiffness of these hydrogels.

### Example 9—Swelling, Water Content, and Degradation Tests

The weight of each sample (n=9) shown in Table 1 was measured following photo-crosslinking and after 1 h, 2 h, 6 h, 1 day, 2 days, 3 days, 1 week, 2 weeks, 3 weeks and 4 weeks incubation in phosphate-buffered saline (PBS) at 37° C. Swelling ratio was then calculated according to Eq. 1, where W₀ is the weight of the sample just after photo-crosslinking and W₁ is the final weight of the sample after 24 h incubation.

$$Swelling ratio ( \% ) = \frac{{W}_{1} - {W}_{0}}{{W}_{1}} \times 100 \qquad ( Eq . 1 )$$

After 24 h incubation at 37° C. in Dulbecco's phosphate-buffered saline (DPBS), the samples were then freeze-dried and weighed. The water content was calculated according to Eq. 2, where W₁ is the weight of the sample after 1-day incubation in PBS and W₂ is the weight of the dried sample.

$$Water content ( \% ) = \frac{{W}_{2} - {W}_{1}}{{W}_{2}} \times 100 \qquad ( Eq . 2 )$$

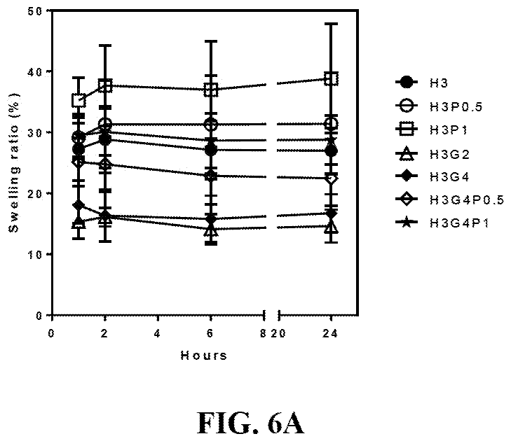

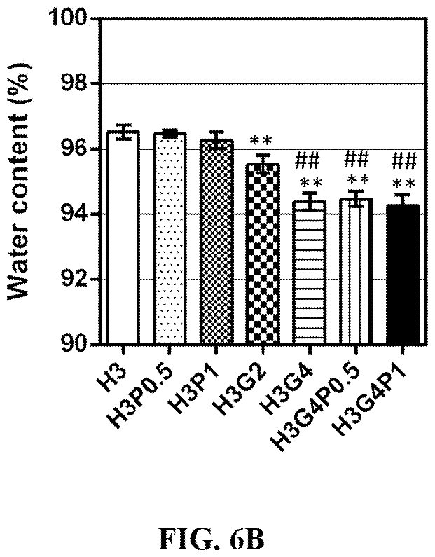

As shown in FIG. 6A, swelling ratios were found to be between about 10% and 40%. The formulations containing 2% or 4% GelMA showed lower swelling ratios compared with MeHA only. The formulations containing 0.5 or 1% PEGDA showed lower swelling ratios compared with MeHA only. The formulations containing PEGDA and GelMA showed similar swelling ratios compared with MeHA only. These results showed that the hydrogels can be used as a swelling-controlled system for ocular drug delivery. Due to mesh size increasing, drugs can be released from the sealant to the ocular surface. The swelling properties of the sealant compositions of the disclosure can be finely turned by modifying polymer concentrations and ratios, that can modify the drug release. Different types of drugs can be loaded in the hydrogel sealant compositions. Non-limiting examples of drugs that can be loaded include antimicrobial drugs, anti-inflammatory drugs, and growth factors.

High water content provided greater oxygen transmission for the cornea and thus, a better biocompatibility. As shown in FIG. 6B, water content of the sealant compositions was found to be between about 94 and 97%, which is even higher than the physiologic water content of the human cornea (i.e., the human cornea has a water content of about 78%).

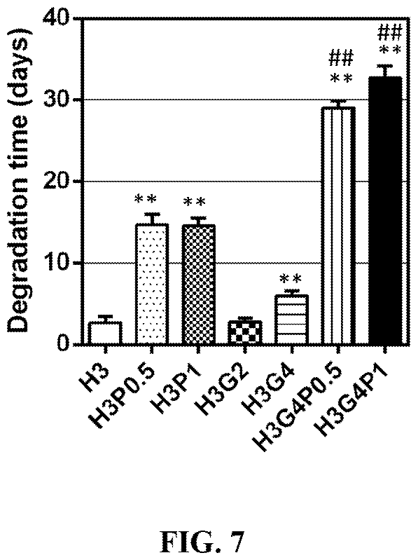

In vitro degradation rates of each formulation were assessed. All samples were incubated in DPBS supplemented with 2 U/mL of hyaluronidase and collagenase I, which are two enzymes that are naturally present in the eye. The presence of GelMA and, most importantly, PEGDA decreased the degradation rate of the sealant as shown in FIG. 7.

### Example 10—Adhesive Properties of Different Sealant Formulations

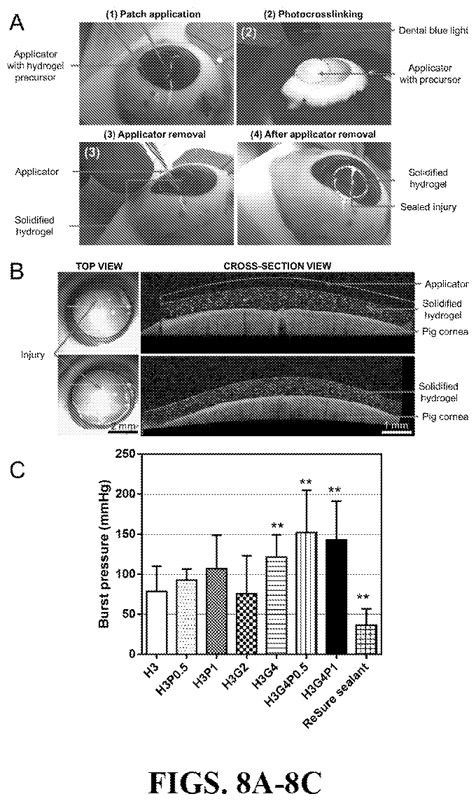

In order to correctly heal ocular injuries, an effective ocular sealant must have high adhesive properties, especially in a wet or aqueous environment. The adhesive properties of the different sealant compositions shown in Table 1 were assessed by using an ex vivo model of pig eyeballs. A 4 mm linear incision was created in the cornea of each pig eyeball. An infusion cannula was placed in the eye to reproduce the physiologic intraocular pressure (TOP) and to monitor success of a proper incision. Once incision success was confirmed (i.e., once leaking of fluid from the incision was visible), the sealant was applied on the incision (FIGS. 8A-B). IOP was then increased by injecting PBS via the infusion cannula until the hydrogel detached and the incision leaked. A commercially available ocular sealant (i.e., ReSure®) sealant was used as a control. The pressure values held by each group were recorded using a pressure sensor. Results demonstrated a higher pressure for all sealant compositions of the disclosure compared to ReSure®, showing that the sealants of the disclosure exhibited higher adhesive properties on the ocular surface (FIG. 8C). Results showed that the addition of 4% GelMA significantly increased the adhesive properties of the MeHA sealant in an ex vivo pig corneal injury.

### Example 11—Drug-Eluting Properties of Different Sealant Formulations

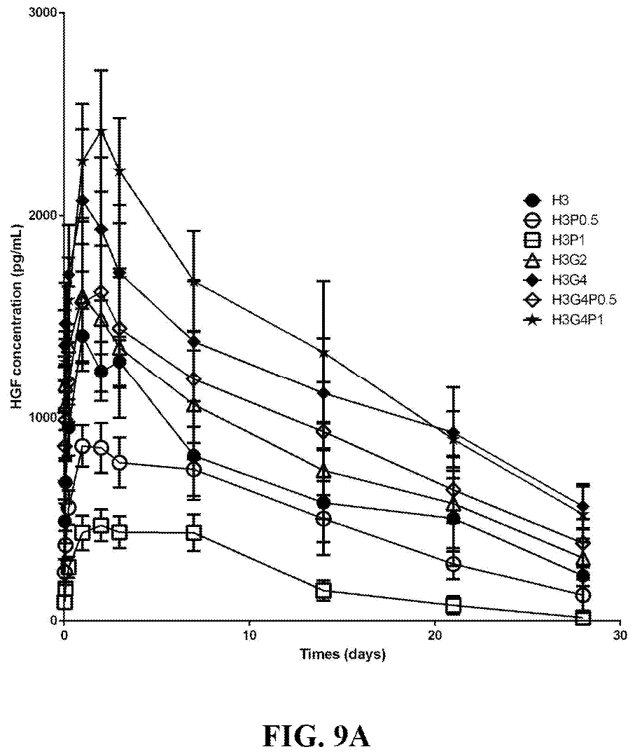

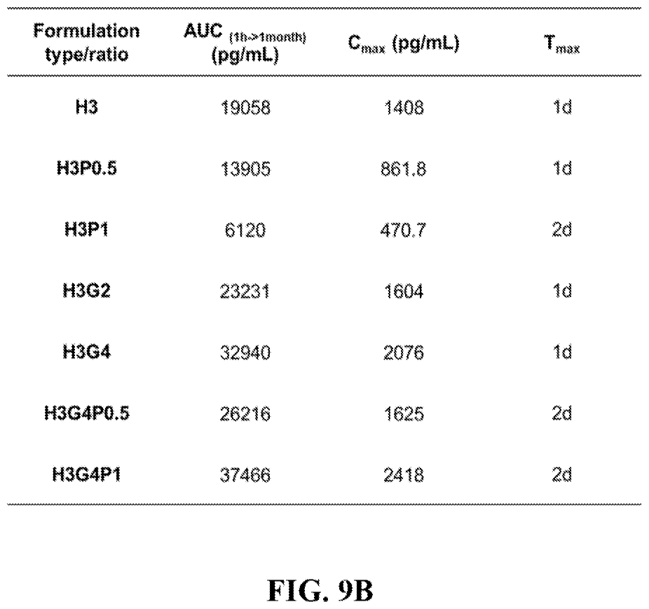

In order to study the healing properties of the sealant compositions, hepatocyte growth factor (HGF) was loaded in the formulations shown in Table 1. Samples containing 500 ng/mL of HGF were prepared as described in Example 8. Then, samples were incubated in PBS supplemented with 0.1% Bovine Serum Albumin (BSA) for 1 month. BSA was used to stabilize HGF. The release of HGF was assessed at different time points (1 h, 2 h, 6 h, 1 day, 2 days, 3 days, 1 week, 2 weeks, 3 weeks, 1 month), using enzyme-linked immunosorbent assay (ELISA). As shown in FIG. 9A, HGF release varied according to the formulation, with a highest release (AUC and Cₘₐₓ) for the formulation H3G4P1. Moreover, the release peak (Tₘₐₓ) was between 1 and 2 days according to the formulation. FIG. 9B shows the AUC, Cₘₐₓ, and Tₘₐₓ, which are standard measurements in pharmacokinetics, for the different sealant formulations. AUC or area under the curve is defined as the total drug concentration released over time. Cₘₐₓ indicates the maximum or peak concentration that a drug achieves after being delivered. Tₘₐₓ is the time at which Cₘₐₓ is observed.

### Other Embodiments

It is to be understood that while certain embodiments have been described within the detailed description, the present disclosure is intended to illustrate and not limit the scope of any embodiment defined by the scope of the appended claims. Other aspects, advantages, and modifications are within the scope of the appended claims.

What is claimed is:

1. A sealant composition, comprising:
methacrylated hyaluronic acid (MeHA);
gelatin methacryloyl (GelMA);
optionally, polyethylene glycol diacrylate (PEGDA); and
a visible, light-activated photoinitiator.

2. The sealant composition of claim 1, wherein the sealant composition does not comprise a hydrolyzing enzyme.

3. The sealant composition of any one of claims 1-2, wherein the sealant composition does not comprise a glycosidase hydrolyzing enzyme.

4. The sealant composition of any one of claims 1-3, wherein the visible light-activated photoinitiator comprises triethanolamine, N-vinylcaprolactam, riboflavin, 2-hydroxy-4′-(2-hydroxyethoxy)-2-methylpropiophenone, Eosin Y disodium salt, 4,6-trimethylbenzoylphosphinate, triethanol amine, dl-2,3-diketo-l,7,7-trimethylnorcamphane (CQ), 1-phenyl-1,2-propadione (PPD), 2,4,6-trimethylbenzoyl-diphenylphosphine oxide (TPO), bis(2,6-dichlorobenzoyl)-(4-propylphenyl)phosphine oxide, 4,4′-bis(dimethylamino)benzophenone, 4,4′-bis(diethylamino)benzophenone, 2-chlorothioxanthen-9-one, 4-(dimethylamino)benzophenone, phenanthrenequinone, ferrocene, diphenyl(2,4,6 trimethylbenzoyl)phosphine oxide/2-hydroxy-2-methylpropiophenone (50/50 blend), dibenzosuberenone, (benzene) tricarbonylchromium, resazurin, resorufin, benzoyltrimethylgermane, derivatives thereof, or any combination thereof.

5. The sealant composition of any one of claims 1-4, wherein the visible light-activated photoinitiator comprises a mixture of triethanolamine, N-vinylcaprolactam, riboflavin, 2-hydroxy-4′-(2-hydroxyethoxy)-2-methylpropiophenone, and Eosin Y disodium salt.

6. The sealant composition of any one of claims 1-5, wherein the visible light-activated photoinitiator is activated upon exposure of light having a wavelength between about 420 nanometers (nm) to 550 nm.

7. The sealant composition of any one of claims 1-6, wherein the MeHA comprises glycidyl methacrylate-hyaluronic acid.

8. The sealant composition of any one of claims 1-7, wherein the MeHA is present in the sealant composition at a concentration between about 1% and 3% weight per volume (w/v).

9. The sealant composition of any one of claims 1-8, wherein the GelMA comprises methacrylamide substitution and methacrylate substitution.

10. The sealant composition of any one of claims 1-9, wherein a ratio of methacrylamide substitution to methacrylate substitution is between about 80:20 and 99:1.

11. The sealant composition of any one of claims 1-10, wherein the GelMA is present in the sealant composition at a concentration between about 2% and 4% (w/v).

12. The sealant composition of any one of claims 1-11, wherein the GelMA is present in the sealant composition at a concentration between about 0.5% and 1% (w/v).

13. The sealant composition of any one of claims 1-12, wherein the GelMA has a degree of methacryloyl substitution between about 30% and 85%.

14. The sealant composition of any one of claims 1-13, wherein the sealant composition further comprises a therapeutic agent.

15. The sealant composition of claim 14, wherein the therapeutic agent comprises an antibiotic, an anti-inflammatory drug, a growth factor, or any combination thereof.

16. The sealant composition of claim 15, wherein the growth factor comprises epithelial growth factor, fibroblast growth factor, nerve growth factor, hepatocyte growth factor, or any combination thereof.

17. The sealant composition of any one of claims 1-16, wherein the sealant composition has a swelling ratio ranging from about 25% to about 35%.

18. The sealant composition of any one of claims 1-17, wherein the sealant composition has a viscosity ranging from about 0.5 Pascal-seconds (Pa·s) to about 200 Pa·s.

19. The sealant composition of any one of claims 1-18, wherein the sealant composition has a burst strength of about 100 millimeters of mercury (mmHg) to 150 mmHg.

20. The sealant composition of any one of claims 1-19, wherein the sealant composition has a degradation rate of about 30 days to 35 days.

21. The sealant composition of any one of claims 1-20, for use in a repair of an ocular surface injury.

22. The sealant composition of any one of claims 1-21, wherein PEGDA is present at a concentration between about 0.5% and 1% (w/v).

23. A method of treating an ocular surface injury in an eye of a subject, the method comprising:
applying the sealant composition of any one of claims 1-21 to an applicator;
placing the applicator containing the sealant composition on a surface of the eye of the subject, wherein the surface has or is suspected of having the ocular surface injury; and
photo-crosslinking the sealant composition by exposing the sealant composition to a visible light.

24. The method of claim 23, wherein the applicator is a contact lens.

25. The method of any one of claims 22-24, wherein the visible light has a wavelength of about 400 nanometers (nm) to 800 nm.

26. The sealant composition of any one of claims 22-25, wherein the ocular surface injury is a corneal injury or a scleral injury.

27. The sealant composition of any one of claims 22-26, wherein the corneal injury is a corneal full-thickness laceration or a corneal full-thickness perforation.

28. The sealant composition of any one of claims 22-27, wherein the ocular surface injury has a depth that is greater than about 350 microns.

29. The sealant composition of any one of claims 22-28, wherein the ocular surface injury extends into a Descemet's membrane or a corneal endothelium.

30. The sealant composition of any one of claims 22-29, wherein the ocular surface injury is a full thickness laceration or a full thickness perforation.
